(19) United States
(12) Patent Application Publication
(10) Pub. No.: US 2020/0360080 A1
(43) Pub. Date: Nov. 19, 2020

(54) APPARATUS AND METHOD OF OCCLUDING A VESSEL BY ABLATION

(71) Applicant: Cook Medical Technologies LLC, Bloomington, IN (US)

(72) Inventors: Rune Paamand, Humlebaek (DK); Per Elgaard, Haslev (DK)

(73) Assignee: Cook Medical Technologies LLC, Bloomington, IN (US)

(21) Appl. No.: 16/821,376

(22) Filed: Mar. 17, 2020

(30) Foreign Application Priority Data
Mar. 18, 2019 (GB) 1903659.9

(51) Int. Cl. A61B 18/14

(52) U.S. Cl. CPC A61B 18/1492; A61B 2018/00404

(57) ABSTRACT

A vessel ablation system includes apparatus and a process in which during a first period t₁ energy is applied at a low power of 3-5 Watts, and determines if the impedance is between 80-125 Ω. Subsequent to this, in a period t₂, power is ramped up linearly from 0 to 40 Watts over a 10 second period. Once the desired optimum power output is reached, the system holds power level for a further period (t₃) of around 20 seconds. The system then enters an after burn stage in which power is reduced to around 10 Watts for a given period t₄. Throughout periods t₂ and t₃, the control unit monitors impedance and if at any stage this exceeds 150 Ω, step t₂ or t₃ is curtailed and the after burn step is immediately implemented. The system provides for partial retraction of the electrode (16) by around 5 millimetres before the sequence is repeated. This algorithm, it has been found, provides reliable vessel occlusion in a reliable manner that safeguards organ integrity.

### CROSS-REFERENCE TO RELATED APPLICATIONS

This application claims priority to Great Britain Application No. 1903659.9, filed Mar. 18, 2019. The contents of Great Britain Application No. 1903659.9 are incorporated by reference in their entirety.

### TECHNICAL FIELD

The present invention relates to apparatus and a method for occluding or closing a vessel by ablation, preferably by the application of radio frequency energy within the vessel.

### BACKGROUND ART

There are numerous medical conditions when it is desired or necessary to close a body vessel, including for instance in the treatment of aneurysms, varicose veins, arteriovenous malformations, arteriovenous fistulas, for starving organs of oxygen and nutrients for instance in the treatment or containment of cancerous growths, and so on.

Several techniques are known and in use for closing or occluding such body vessels. Traditionally, vessels have been closed by means of external ligation, which generally must be carried out by an open surgery procedure, with its associated risks, inconvenience and long patient recovery times. Other more recent methods aim to use an endoluminal procedure to insert into the vessel or organ one or more occlusion devices, such as a metal framed occluder, coils, pellets or the like, able to obstruct the flow of blood in the vessel.

It is also known to seek to constrict a vessel by endoluminal ablation, causing contraction of the vessel and/or coagulation of blood to form a blood clot in the vessel. A technique that has been considered suitable is RF ablation, in which an electrical terminal is fed endoluminally into the vessel and an electrical pulse at RF frequencies applied to the electrical terminal. The conductivity of blood and/or the vessel tissues causes localised heating. This heating can be used to cause damage to the tissue (intima) of the vessel wall, resulting in vessel contraction. In other devices RF ablation heats the surrounding blood, causing this to coagulate around the electrical terminal and form a blood clot which blocks the vessel. Vessel occlusion by blood clotting may also cause reaction of the vessel tissues.

Two types of RF ablation apparatus are generally contemplated in the art, the first being a monopolar system having an elongate active terminal and a passive/dispersive pad. The active terminal is designed to be fed endoluminally into the patient's vessel, while the passive pad is positioned against the person's outer body, as close as practicable to the active terminal. Electrical energy applied to the active terminal will pass by conduction through the patient to the passive pad. There will be localised heating at the active terminal, which effects the desired ablation.

A problem with monopolar systems is that it can be difficult to control the extent of damage to surrounding tissues and organs, as well as to the vessel wall. This risks damaging the vessel to the point of rupture, as well as causing potential irreversible damage to neighbouring organs.

Another RF ablation system uses a bipolar arrangement, in which an elongate electrical element includes two terminals disposed at a distal end of the electrical element and spaced longitudinally from one another. Current passes between the two terminals through the surrounding blood, causing localised heating and coagulation of the blood. A bipolar system has been considered to provide more localised heating but this can be more intense than with a monopolar system.

A number of problems have been identified during testing and use of such systems. A first lies with the retraction of the electrical terminal from the vessel at the end of the ablation process. In a system that ablates the vessel wall to cause its contraction, the electrical terminal can become attached to the vessel wall tissue, with the risk of tearing and rupturing the vessel wall. In a system that ablates the surrounding blood to generate a blood clot in the vessel, there is the risk that the blood clot is dragged with the electrical element and that the occlusion of the vessel is as a consequence lost. There is also the risk of leaving an opening in blood clot where the electrical terminal resided, which can result in incomplete occlusion and the risk of recanalization.

Another problem lies with damage caused to the vessel and surrounding organs by the heating process. There is a balance, therefore, between applying sufficient heat to cause satisfactory ablation yet limiting the extent and reach of heating so as to seek to minimise any damage to outlying organs, including the vessel itself in the case of occlusion by blood clot formation.

Often, systems and method must compromise between these factors.

Examples of prior art devices and methods can, for instance, be found in US-2015/0289930, US-2017/0202556, U.S. Pat. No. 9,161,813, US-2016/0058492, U.S. Pat. No. 6,796,981, U.S. Pat. No. 5,827,271 and US-2016/0045248.

### SUMMARY OF THE INVENTION

The present invention seeks to provide improved apparatus and method for occluding or closing a body vessel.

According to an aspect of the present invention, there is provided vessel occlusion apparatus including:

an RF energy source configured to output an RF signal to an electrode deployable endoluminally within a vessel of a patient; and

a control unit, through control of the RF energy source in order to carry out vessel occlusion, configured to:

a) determine an impedance through the electrode and, while the measured impedance is no more than substantially 150 Ω:

(i) ramp up power of the RF signal to a predetermined first power level of substantially 40 watts over a first period of no more than substantially 10 seconds;

(ii) maintain the power of the RF signal at the predetermined first power level for a given second period; and

(iii) output the RF signal at a second, lower power level for a predetermined third period in response to the measured impedance reaching or exceeding substantially 150 Ω.

The inventors have discovered that optimal ablation of a vessel can be optimised with the disclosed process or algorithm steps. Specifically, the system and method are able to provide reliable vessel closure, even in a wide variety of vessel diameters and flow types, yet minimise the risk of collateral damage to nearby organs and tissues.

Advantageously, the control unit is configured to apply power at the second power level with the electrode in the same position as during the first period.

Preferably, the control unit is configured to repeat the occlusion process with the electrode moved proximally in the vessel a given distance. Such repetition can close off any remaining apertures in the vessel following the first heating stage, even any left by the electrode itself. The given distance is preferably substantially 5 millimetres.

Advantageously, the control unit is configured to ramp up power to the predetermined first power level from substantially zero power, and preferably substantially linearly. Such ramping up of the power can lead to an effective and not excessive delivery of ablation energy for a variety of vessel sizes and flow types.

It is preferred that the second period is no more than substantially 20 seconds.

The second power level is substantially 10 watts in the preferred embodiment, and may be supplied for a period of substantially 5 seconds.

In practical embodiments, the control unit is operable to carry out a check prior to application of heating energy at power level between 3 and 5 watts and to determine whether impedance is between 80-125 Ω.

According to another aspect of the present invention, there is provided a non-transitory computer readable storage medium storing computer instructions, or an algorithm therefor, the computer instructions executable by a control unit to perform a power control algorithm for vessel occlusion through control of an RF energy source, the instructions comprising:

a) instructions to measure an impedance through an electrode deployable endoluminally within a vessel of a patient, and, while the measured impedance is no more than substantially 150 Ω:

(i) instructions to ramp up a power of an RF signal to a predetermined first power level of substantially 40 watts over a first period of no more than substantially 10 seconds, the RF signal output to the electrode by the RF energy source;

(ii) instructions to maintain the power of the RF signal at the predetermined first power level for a given second period; and

(iii) instructions to output the RF signal at a second, lower, power level for a predetermined third period in response to the measured impedance reaches or exceeds substantially 150 Ω.

Advantageously, power is applied at the second power level with the electrode in the same position as during the first period.

The computer instructions, may include repeating the occlusion process with the electrode moved proximally in the vessel a given distance.

The given distance is preferably substantially 5 millimetres.

Power may be ramped up to the predetermined first power level substantially linearly.

The second period is preferably no more than substantially 20 seconds. The second power level may be substantially 10 watts and may be supplied for a period of substantially 5 seconds.

The computer instructions preferably include a check prior to application of heating energy at power level between 3 and 5 watts and to determine whether impedance is between 80-125 Ω.

According to another aspect of the present invention, there is provided a method of occluding a vessel of a patient; the method including:

a) measuring, with a control unit, an impedance through an electrode deployable endoluminally within the vessel, and, while the measured impedance is no more than substantially 150 Ω:

(i) ramping up, with the control unit via an RF energy source outputting an RF signal, a power of the RF signal to a predetermined first power level of substantially 40 watts over a first period of no more than substantially 10 seconds;

(ii) maintaining, with the control unit via the RF energy source, the power of the RF signal at the predetermined first power level for a given second period; and

(iii) outputting the RF signal at a second, lower, power level for a predetermined third period in response to the measured impedance reaching or exceeding substantially 150 Ω.

Other aspects and advantages of the teachings herein will become apparent to the skilled person from the disclosure that follows.

### BRIEF DESCRIPTION OF THE DRAWINGS

Embodiments of the present invention are described below, by way of example only, with reference to the accompanying drawings, in which:

FIG. 1 is a side elevational view of an example of endoluminal energy delivery device;

FIG. 2 is an enlarged side elevational view of the conductive electrode tip of the device of FIG. 1;

FIG. 3 is a schematic view of the main components of an embodiment of vessel occlusion apparatus incorporating the teachings herein;

FIGS. 4A to 4C are schematic cross-sectional views showing preferred stages of operation of the apparatus of FIG. 3 and device of FIGS. 1 and 2;

FIG. 5 is a graph showing the preferred embodiment of power control algorithm operated by the apparatus of FIG. 3; and

FIG. 6 is a flow chart showing the preferred embodiment of power control algorithm operated by the apparatus of FIG. 3.

### DESCRIPTION OF THE PREFERRED EMBODIMENTS

Described below are preferred embodiments of vessel occlusion apparatus and method of occluding a vessel. Occlusion is preferably by ablation with energy delivered at RF frequencies intended to heat blood in the vessel in order to cause it to clot, thereby forming an occluding plug. The apparatus and method may also be used to ablate vessel tissue so as to cause the vessel to close. The preferred system causes vessel closure by a combination of thrombus generation and vessel closure.

The described embodiments are to a monopolar RF ablation system, in which an electrode is deployed endoluminally into a patient from a percutaneous entry point. A second electrode, typically the cathode, is in the form of a pad that is kept outside the patient and positioned against the patient's skin, as close as practicable to the location of the vessel to be ablated, that is to the terminal disposed in the patient, the latter acting as an anode. Electrical energy applied to the anode terminal will pass by conduction through the patient to the cathode pad, causing localised heating at the anode terminal, which effects the desired ablation.

It will be appreciated that when the device is used to heat vessel tissue, current passed to the anode terminal will heat and cause contraction and occlusion of the vessel.

The skilled person will appreciate that the teachings herein are equally applicable to a bipolar system, in which the electrode carries both the anode and cathode terminals at its distal end, separated by a length of insulation (such as a sleeve).

The teachings herein can be used for any of the previously stated medical indications, as well as others such as embolization of arteries in relation to gastric bleeding, solid organ and muscle trauma and tumours (for example prior to radio/bead embolization).

The structure of the embolization anode electrode described herein can enable it to be used also as a guide wire, enabling the device to achieve super-selective embolization of very small vessels. The device does not require a micro catheter to track to the treatment target, making it suitable for very small vessels, for example of a diameter of no more than 2.5 mm, such as peripheral and visceral arteries.

The electrode tip is preferably flexible, allowing it to navigate through the tortuous vasculature. It is preferred that the electrode has a curved shape, which it has been found can optimise blood heating and coagulation in a vessel.

In the description that follows the term “proximal” is representative of a location closer to the physician in use, while the term “distal” refers to a positon further away from the physician and in practice within the patient, in this case at or towards the endoluminal treatment site.

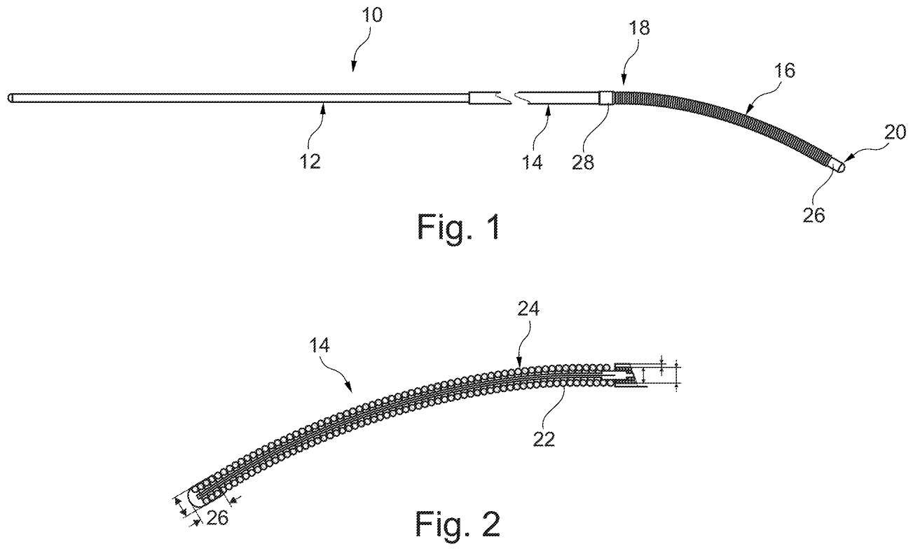

Referring to FIG. 1, this shows and example of RF anode electrode structure 10 which includes a proximal portion 12, an intermediate portion 14 and an energy delivery electrode tip 16 extending from the intermediate portion 14. The electrode tip 16 has a proximal tip end 18 and a distal tip end 20.

The intermediate portion 14, as well as the proximal portion 12, extend in a generally linear (first) direction when unbiased, while the electrode tip 16 extends in a different (second) direction, the electrode tip end 20 preferably being offset from the proximal electrode tip end 18 relative to the first direction by between 10 to 50% of the tip length, in this embodiment by substantially 20% of the tip length. This can enable the electrode tip 16 to contact a vessel wall at two points, in practice at or near the proximal 18 and distal 20 tip ends.

In the example shown, the electrode tip 16 achieves the deviation from the axis of the remainder of the structure by gentle curvature, although it is not excluded that this could be achieved by a bend at the proximal end of the tip 16.

FIG. 2 is an enlarged view of the electrode tip 14 of FIG. 1. The electrode 10 includes a core 22 that extends from the proximal end 18 to the distal end 20 of the electrode tip 16. In practice, the core 22 extends for the whole length of the electrode 10, to its most proximal end outside the patient. The core 22 is preferably a unitary structure of a single material, for example Nitinol. In other examples, the core 22 may be made of any material that provides good mechanical properties, such as stainless steel or nickel titanium alloys, and may be made of a plurality of components. The core may have a diameter of around 0.4 millimetres.

Surrounding the core 22 is an electrically conductive element 24, which may be made of any good conductor material, such as platinum, gold, silver or alloys of these. The electrically conductive element 24 may also be made of or include a radiopaque material. In this embodiment, the electrically conductive element 24 is made of platinum with 2-10% by weight tungsten.

In the example shown, the electrically conductive element 24 is in the form of a wire 24 coiled helically around the core 22 and extending from the proximal tip end 18 to the distal tip end 20. Adjacent turns of the coil 24 are preferably tightly wound with space between the coil windings being about 5%±2.5% of the wire diameter, which gives the electrode 16 flexibility.

The tip of the electrode 16 may be in the form of a solder point 26, coupled to the core 22 and the coil 24. Another solder point 28 may be provided at the proximal end 18 of the electrode tip 16.

The skilled person will appreciate that the example electrode device described above has good mechanical properties for advancing and supporting the device in a target vessel as a result of the use of a strong yet flexible core or mandrel 22 and good conductivity through the coil 24.

The mandrel or core 22 may be of uniform diameter along its length or may taper to provide a changing flexibility along it length.

The electrically conductive coil 24 may be coated with a heat conductive material. This is not necessary but can provide advantages, for example by having non-stick properties.

The intermediate portion 14 may be covered in a sleeve of insulating material, for instance of polymer material such as polyethylene terephthalate (PET) shrink tubing, as fluorinated ethylene propylene (FEP) or polytretraflouroethylene (PTFE).

Advantageously, the distal coil 24 has a greater flexibility than the core 22. This allows the distal coil 24 to follow the curve of the core 22. The electrode element 16 may be curved at a constant radius of curvature over its length. The element 16 is preferably also able to be reshaped by the physician during the course of a medical intervention, for example to tighten its radius of curvature or to give it a variable radius of curvature.

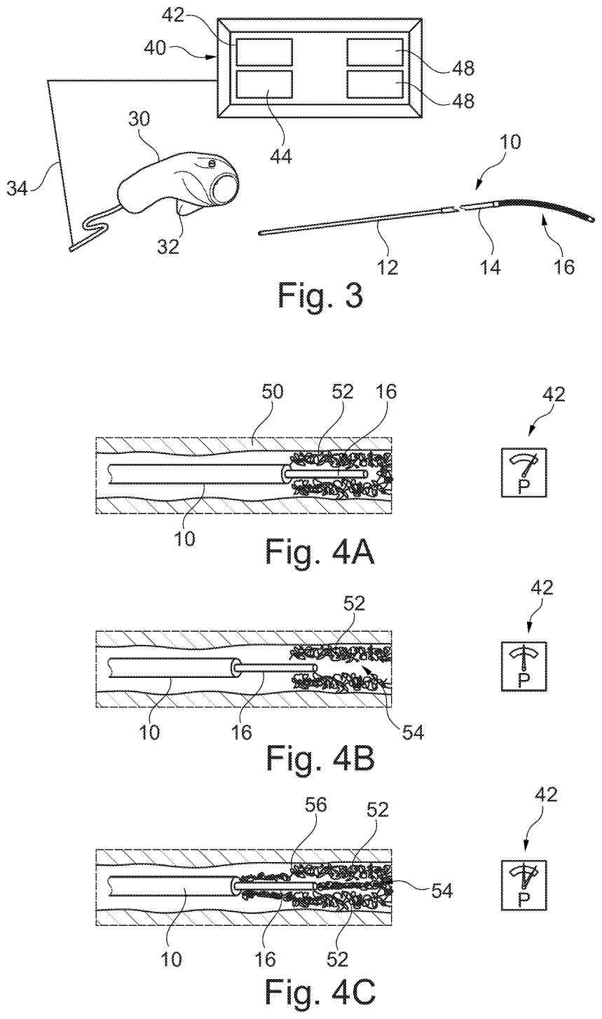

Referring now to FIG. 3, this shows the principal components of the ablation apparatus, being in this example, as described above, a monopolar system. In addition to the elements shown in FIG. 3, the system includes a conductive cathode pad or other element (not shown in FIG. 3) designed to be kept outside the patient and typically placed against the patient's body at a location close to the treatment site. As cathode pads for this purpose are known in the art, it is not described further herein.

The apparatus includes a control unit 40 that comprises a power supply unit 42 configured to output electrical energy at RF frequencies, that is as an RF energy source; a controller 44, typically a microprocessor; an impedance sensor 46; and a memory 48, that may be any suitable non-transitory computer readable storage medium storing computer instructions, of which many examples are known in the art.

The impedance sensor 46 is configured to determine the impedance of the circuit formed by the anode and cathode electrodes of the system. It may in some embodiments also include a temperature sensor configured to sense temperature at the electrode tip 16.

The power supply unit may be a standard 50 W RF generator providing a sine output at a frequency of approximately 500 kHz.

The apparatus also includes a handle 30 with a trigger 30, the handle in use being electrically connected to the control unit 40 via an electrical wire or cable 34. When it is desired to ablate a vessel, the electrode element 10 is inserted into the patient's vasculature, often from a remote percutaneous entry point. The proximal end 12 of the element 10 is fitted into the handle 30 to make an electrical connection between the electrode 16 and the control unit 40, as well as any other associated devices such as temperature probes.

The control unit 40 then applies RF power to the vessel though the electrode 16 and the associated cathode pad (not shown in the Figures), in a sequence and according to an algorithm of the type described below. The electrode element 10 is designed to be removed from the patient following the procedure.

As indicated above, optimal vessel ablation should control the power output to prevent charring and unnecessary heating, while still ensuring that good and reliable vessel closure is achieved. It is known in such systems to obtain an indication of blood clotting by sensing an impedance shift in the electrical circuit and it is also known to carry out staged ablation steps, as disclosed for example in the applicant's earlier applications EP-2,939,629 and EP-3,153,122. In these are earlier applications, an ablation method is taught in which the endoluminal electrode is partially withdrawn and more energy is then applied to seal off any potential channel within the blood clot that has been formed.

It has also be found that excessive ablation energy can result in a blood clot adhering too firmly to the electrode and subsequent difficulties in removing the electrode from the blood vessel. While some systems have developed a severable electrode tip, it is considered that this is not ideal as it leaves a foreign object in the patient. It has been found that this problem can be mitigated by stopping power delivery milliseconds after detection of a shift in impedance in the circuit, this being a feature provided for in the preferred embodiments described herein.

In general, it is preferable to use as little ablation energy as possible in a treatment in order to minimise the risk of heat damage to surrounding tissue and other organs. The preferred embodiment achieves this by generating sufficient power to initiate a thrombosis cascade in a relatively short time in order to limit heat conduction. As the target vessel can vary substantially in size, from sub-millimetre to 2½ mmm or so, the system advantageously adjusts to allow delivery of power monitored by a reliable impedance threshold. Power can be adjusted either by varying the actual power output of the unit 42 or to use a cut-out relay to duty-cycle the available power. The duty cycle frequency of this embodiment is kept low enough to limit the bandwidth of the output spectrum to above 200 kHz, and is effectively a square wave amplitude modulated signal.

The preferred embodiment described below has three primary characteristics to the power output by the power supply unit 42. The first is a ramp up in power up to a threshold, whereupon power is kept substantially uniform for a maximum period of time and thereafter reduced to an after-burn period. The concept is to provide a relatively rapid ramp up in power to generate relatively rapid occlusion of the vessel but not to cause power to exceed a predetermined optimum maximum power level, which the inventors have identified as in the region of 40 Watts.

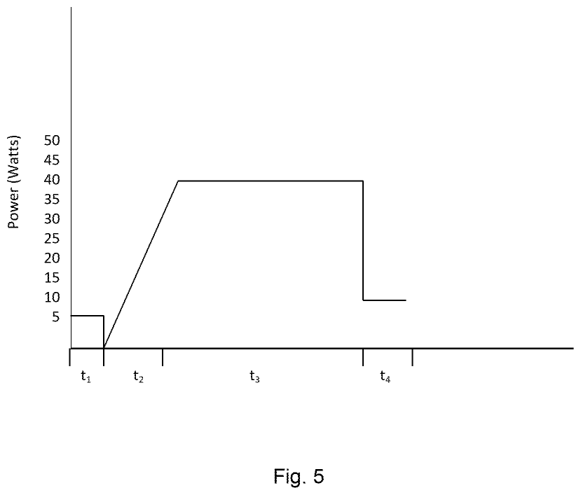

With reference to FIG. 5, this shows a graph depicting the preferred power control algorithm and method of occluding a vessel in accordance with the teachings herein.

It is preferred that the system begins by carrying out what could be described as a sanity check, that is a verification that the system is correctly set up and the vessel suitable. Thus, during period t₁ the system applies energy at a low output power, for example between 3-5 Watts, and determines the impedance of the circuit. If the measured impedance is between 80-125 Ω, in this embodiment, it is determined that the apparatus is set up correctly and that effective ablation can be carried out. Subsequent to this, in period t₂ in FIG. 5, power is ramped up, preferably linearly, from 0 to 40 Watts over a 10 second period. In some embodiments, this could be less than 10 seconds, for example for around 2-3 seconds or any period up to 10 seconds.

Once the desired optimum power output of substantially 40 Watts has been reached, the system holds power level for a further period t₃, which in the example shown is around 20 seconds. In practice, period t₃ should be at least as long as the ramp time of period t₂, but most preferably twice the ramp time t₂. After period t₃, beyond which the inventors have identified that no improvement in vessel ablation is likely, the system enters an “after burn” stage in which power is reduced, in this example, to around 10 Watts for a period t₄. This period t₄ is preferably shorter than periods t₂ and t₃ and preferably about half the ramp time t₂.

Throughout periods t₂ and t₃, the control unit and in particular the impedance sensor 46 thereof monitors the impedance through the circuit and if at any stage the impedance exceeds 150 Ω, step t₂ or t₃ is curtailed and the after burn step is immediately implemented. Therefore, the power graph of FIG. 5 depicts a maximum ramp and power maintenance sequence that would occur when there is not early cut off power because of a detected shift in impedance.

During stages t₂-t₄ shown in FIG. 5, the electrode 16 is kept in position within the vessel. It is preferred, and in accordance with the applicant's earlier European applications referred to above, that after the sequence shown in FIG. 5, the system provides for partial retraction of the electrode 16, for example by around 5 millimetres or so, before the sequence of FIG. 5 is repeated. Retraction of the electrode 16 can be effected by a suitable actuator mechanism in the handle 30 or by the physician.

Referring now briefly to FIGS. 4A to 4C, these depict a two stage vessel embolization procedure of the type referred to above. These Figures show the electrode tip 16 being in a straight configuration which, while a practical embodiment for the teachings herein, would preferably be replaced by a curved electrode of the type shown in FIGS. 1 and 2. With reference to FIG. 4A, upon generation of power during the period t₂ or periods t₂ and t₃ (in dependence upon the moment of detection of impedance shift) blood forms a coagulum as a result of heating caused by the RF energy applied through the electrode 16. This will be detected by a shift in impedance picked up by the impedance sensor 46. The electrode 16 can be partially withdrawn from the formed blood clot 52, as depicted in FIG. 4B and relatively easily in light of the after burn stage during period t₄, which prevents the coagulated blood adhering too tightly to the electrode 16.

FIG. 4C shows the effect of repetition of the sequence of FIG. 5, in which further embolization of blood occurs within any gap 54 left after the first stage procedure and a greater volume of coagulated blood 56 is generated during the second stage procedure. As at this stage the vessel will be occluded or substantially occluded, it is expected that a repetition of the sequence of FIG. 5 will be very quick and likely to take less than 15 seconds in totality. This will be dependent, of course, upon the nature of the blood vessel, particularly its size and blood flow rate.

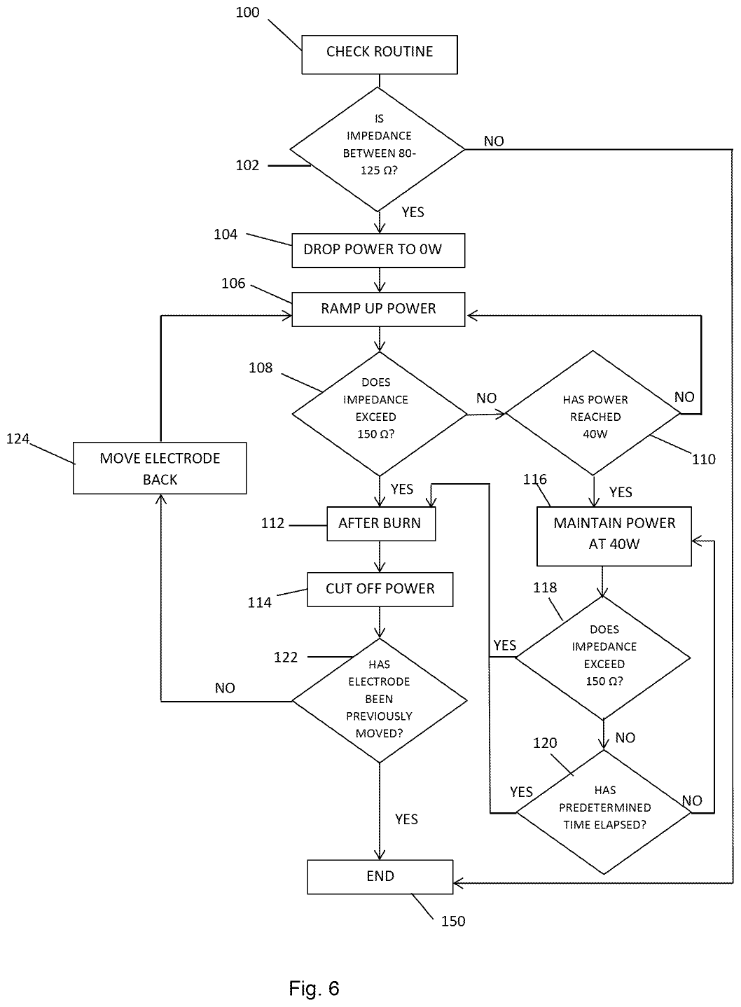

Referring now to FIG. 6, this shows a flow chart of the preferred method of occluding a vessel, carried out within the control unit 40 of the apparatus depicted in FIG. 3. It will be appreciated that the process of FIG. 6 will be typically implemented as an algorithm stored in memory 48, typically any suitable non-transitory computer readable storage medium that stores the computer instructions executable by the microprocessor 44 of the control unit 40 through control of the RF energy source 42.

The algorithm commences at step 100, in which a check routine, or sanity check is performed, in accordance with that depicted at period t₁ in FIG. 5. In this routine, RF energy is applied at a low power between 3-5 Watts and it is determined whether the impedance in the circuit is between 80-125 Ω, at step 102. If the measured impedance falls outside this range, the control algorithm comes to an end, at step 150. On the other hand, if measured impedance during the check routine is between 80 and 125 Ω, the algorithm proceeds to step 104, at which power output is cut to zero Watts. It will be appreciated that in some embodiments power may be maintained at the low output of the check routine or any other low output, although it is preferred that it is cut completely.

At step 106, the system then begins ramping up power to the electrode 116, preferably though not essentially in linear manner. During this stage, the impedance sensor 46 continues to monitor (step 108) the impedance of the circuit and if it is detected the impedance does not exceed the predetermined maximum, in this example of 150 Ω, it is then determined, at step 110 whether the power has reached 40 Watts. If power has not yet reached the threshold, the routine continues through to step 106

Should at step 108 it be determined that the measured impedance exceeds the threshold the routine proceeds to step 112, at which the after burn stage is carried out, that is with RF energy being supplied at a substantially reduced power level, in the preferred embodiment 10 Watts, for a short period, in this example of 5 seconds. After the after burn period, the routine proceeds to step 114, at which all power to the electrode 16 is cut off.

Returning to step 110, if it is determined that the maximum power has been reached, the routine proceeds to step 116, at which power is maintained at a steady level, preferably 40 Watts. During this period the routine determines, at step 118, whether the measured impedance exceeds the 150 Ω threshold. If it does, the routine passes to the after burn stage 112. On the other hand, if the impedance threshold has not been exceeded, the routine passes to step 120 at which it is determined whether the predetermined time (t₃) has elapsed. If it has not, the routine continues with step 116. On the other hand, if the predetermined time has elapsed, the routine passes to the after burn step 112.

Once power has been cut off at step 114, the routine passes to step 122, at which it is determined whether the electrode 16 has been previously moved in the vessel. If it has not, the electrode 16 is moved back, at step 124 by a predetermined distance, which may be in the region of 5 mm, and the routine then returns to step 106. If, on the other hand, it is determined at step 116 that the electrode has previously been moved, the routine proceeds to step 150, at which the routine comes to an end and all power is cut off.

The preferred clinical protocol for use with the described ablation control algorithm will include the following steps:

1. To shape electrode tip 16 to a curvature causing a 3 mm deflection normal to the longitudinal axis of the element 10, with optional additional shaping of the electrode 16 by the physician just before the medical intervention.

2. Position the electrode in a target vessel, typically having a diameter of 2.5 mm or less and with the catheter pad preferably more than 1 cm distant from the anode electrode 16.

3. Connect the RF element 10 to the handle 30 and active the control unit 40. If the impedance is not within 80-125 Ω within 1 second, the wire 16 and connections to the control unit 40 are checked to determine that the apparatus has been set up correctly.

4. Execute the power control algorithm (steps 104-120).

5. Once the power control algorithm has been completed, reposition electrode 16 at a proximal section of the target, around 5 mm proximal of the first treatment location;

6. Reactivate power control algorithm and allow to complete (expected time less than 15 seconds).

7. Withdraw electrode 16 and check for reliable occlusion by injection with contrast agent.

8. Repeat the process if the vessel has not fully occluded and check again.

9. Repeat the procedure with a new electrode 16 if full vessel occlusion has not occurred and if this fails abort the procedure.

The ramp up time t₂ during which RF power is ramped up in the electrode has been found to provide an optimum balance between multiple factors experienced during the procedure. Ablation, or embolization, can trigger both a vessel response, namely a denaturation around 50° C. followed by vessel contraction (vasoconstriction) that then triggers a coagulation cascade, and a direct heat related blood coagulation. The inventors have discovered that if the blood is heated too fast, the electrode 16 will char before enough energy has been delivered to the vessel wall, with the risk of coagulating only blood and not initiating vessel response. On the other hand, if heat is applied too slowly, effective occlusion may not occur and there may be risk of too deep vessel damage. A ramp time of 10 seconds has been found to provide a good balance between these factors, although many factors can influence this optimum, including for example vessel diameter, blood flow rate, blood consistency and so on. A ramp time between 1 and 20 seconds, with 10 seconds being preferable, has been found to work effectively.

It will be appreciated that the teachings herein can be used in a bipolar vessel ablation system, in which both the anode and cathode electrodes are deployed endoluminally within the vessel to be treated. Typically, in a bipolar system the energy delivery electrode 26 may include both the anode and the cathode terminals, spaced longitudinally from one another on the device 10 and electrically spaced from one another.

The disclosure in the abstract accompanying this application is incorporated herein by reference.

1. Vessel occlusion apparatus including:
an RF energy source configured to output an RF signal to an electrode deployable endoluminally within a vessel of a patient; and
a control unit, through control of the RF energy source in order to carry out vessel occlusion, configured to:
a) determine an impedance through the electrode and, while the measured impedance is no more than substantially 150 Ω:
(i) ramp up power of the RF signal to a predetermined first power level of substantially 40 watts over a first period of no more than substantially 20 seconds; and
(ii) maintain the power of the RF signal at the predetermined first power level for a given second period;
(b) output the RF signal at a second, lower power level for a predetermined third period in response to the measured impedance reaching or exceeding substantially 150 Ω.

2. Apparatus according to claim 1, wherein the control unit is configured to apply power at the second power level with the electrode in the same position as during the first period.

3. Apparatus according to claim 1, wherein the control unit is configured to repeat the occlusion process with the electrode moved proximally in the vessel a given distance.

4. Apparatus according to claim 3, wherein the given distance is substantially 5 millimetres.

5. Apparatus according to claim 1, wherein the control unit is configured to ramp up power to the predetermined first power level from substantially zero power.

6. Apparatus according to claim 1, wherein the control unit is configured to ramp up power to the predetermined first power level substantially linearly.

7. A system according to claim 1, wherein the second period is no more than substantially 20 seconds.

8. A system according to claim 1, wherein the second power level is substantially 10 watts.

9. A system according to claim 1, wherein the second, lower power level is supplied for a period of substantially 5 seconds.

10. A system according to claim 1, wherein the control unit is operable to carry out a check prior to application of heating energy at power level between 3 and 5 watts and to determine whether impedance is between 80-125 Ω.

11. A system according to claim 1, wherein the first period is at least substantially 1 second.

12. A system according to claim 1, wherein the first period is between about 1 second and about 10 seconds.

13. A system according to claim 1, wherein the first period is between about 2 seconds and about 10 seconds.

14. A system according to claim 1, wherein the first period is substantially 10 seconds.

15. A non-transitory computer readable storage medium storing computer instructions, the computer instructions executable by a control unit to perform a power control algorithm for vessel occlusion through control of an RF energy source, the instructions comprising:
a) instructions to measure an impedance through an electrode deployable endoluminally within a vessel of a patient, and, while the measured impedance is no more than substantially 150 Ω:
instructions to ramp up a power of an RF signal to a predetermined first power level of substantially 40 watts over a first period of no more than substantially 20 seconds, the RF signal output to the electrode by the RF energy source; and
(ii) instructions to maintain the power of the RF signal at the predetermined first power level for a given second period;
(b) instructions to output the RE signal at a second, lower, power level for a predetermined third period in response to the measured impedance reaching or exceeding substantially 150 Ω.

16. Computer instructions algorithm according to claim 15, wherein power is applied at the second power level with the electrode in the same position as during the first period.

17. Computer instructions according to claim 15, including repeating the occlusion process with the electrode moved proximally in the vessel a given distance.

18. Computer instructions according to any one of claims 15, wherein the given distance is substantially 5 millimetres.

19. Computer instructions according to any one of claims 15, wherein power is ramped up to the predetermined first power level substantially linearly.

20. Computer instructions according to any one of claims 15, wherein the second period is no more than substantially 20 seconds.

21. Computer instructions according to any one of claims 15, wherein the second power level is substantially 10 watts.

22. Computer instructions according to any one of claims 15, wherein the second, lower power level is supplied for a period of substantially 5 seconds.

23. Computer instructions according to any one of claims 15, including a check prior to application of heating energy at power level between 3 and 5 watts and to determine whether impedance is between 80-125 Ω.

24. A method of occluding a vessel of a patient; the method including:
a) measuring, with a control unit, an impedance through an electrode deployable endoluminally within the vessel, and, while the measured impedance is no more than substantially 150 Ω:
(i) ramping up, with the control unit via an RF energy source outputting an RF signal, a power of the RF signal to a predetermined first power level of substantially 40 watts over a first period of no more than substantially 20 seconds; and
(ii) maintaining, with the control unit via the RF energy source, the power of the RF signal at the predetermined first power level for a given second period;
(b) outputting the RF signal at a second, lower, power level for a predetermined third period in response to the measured impedance reaching or exceeding substantially 150 Ω.
